(19) United States
(12) Patent Application Publication
(10) Pub. No.: US 2008/0305142 A1
(43) Pub. Date: Dec. 11, 2008

(54) Cell Free Biosynthesis of High-Quality Nucleic Acid and Uses Thereof

(75) Inventors: Yin Chen, Pearland, TX (US); Frederic Kendirgi, Houston, TX (US); Malcolm Skolnick, Houston, TX (US)

Correspondence Address: OSHA & LIANG LLP / CYTOGENIX, TWO HOUSTON CENTER, 909 FANNIN STREET, SUITE 3500, HOUSTON, TX, 77010, US

(73) Assignee: CYTOGENIX, INC., HOUSTON, TX (US)

(21) Appl. No.: 11/792,800

(22) PCT Filed: Dec. 12, 2005
(86) PCT No.: PCT/US2005/045028

§ 371 Date: Jan. 28, 2008

Related U.S. Application Data
(60) Provisional application No. 60/635,418, filed on Dec. 11, 2004.
(60) Provisional application No. 60/680,716, filed on May 13, 2005.
(60) Provisional application No. 60/705,348, filed on Aug. 4, 2005.

(51) Int. Cl. A61F 2/04; C12P 19/34; A01N 43/00; A61K 39/00; A61P 31/12; A61P 31/10; A61K 31/7052; A61K 9/12

(52) U.S. Cl. 424/423; 435/91.5; 504/209; 514/44; 424/45; 424/184.1; 435/286.1

(57) ABSTRACT

The invention provides an improved cell free amplification method capable of producing large quantities of therapeutic-quality nucleic acids and methods of using the synthesized nucleic acid in research, therapeutic and other applications—The methods combine several different state-of-the-art procedures and coordinate their applications to affordably synthesize nucleic acids for therapeutic purposes. It combines in vitro rolling circle amplification, high fidelity polymerases, high affinity primers, and streamlined template specifically designed for particular applications. For expression purposes, the templates contain an expression cassette including a eukaryotic promoter, the coding sequence for the gene of interest, and a eukaryotic termination sequence. Following amplification, concatamers are subsequently processed according to their intended use and may include: restriction enzyme digestion for the production of short expression cassettes (SECs); ligation steps to circularize the SEC (CNAs); and/or supercoiling steps to produce sCNAs. The final product contains nearly non-detectable levels of bacterial endotoxin.

### FIELD OF THE INVENTION

The invention relates to a process for making high quality nucleic acids and the use thereof.

### BACKGROUND OF THE INVENTION

The advent of DNA-based therapeutics in gene transfer, gene therapy and DNA vaccination has increased the demand for large-scale production of DNA that meets stringent quality criteria in terms of purity, potency, efficacy and safety. Because the efficacy and duration of gene expression in target tissue is relatively low, large amounts of DNA are typically needed for such applications.

The current state of the art relies upon the growth of plasmids in bacterial culture and expensive purification techniques for the production of therapeutic quality nucleic acids. Typical plasmid purification procedures from bacteria and other cell sources include methods that use organic, mutagenic and toxic compounds including phenol, ethidium bromide and cesium chloride, and enzymes, such as lysozyme, proteinase K, and RNase A. All these can constitute potential health hazards if injected as contaminants in a DNA-based therapeutic. Such procedures also carry a potential risk of incorporating unintended contaminating transposons and other foreign episomal DNA into the plasmid (Haapa, S., Nuc. Ac. Res., 27(13): 2777-84, 1999). There is also the potential for contamination by residual host cell nucleic acids, other cellular proteins and endotoxins. Such impurities not only minimize the efficiency of DNA uptake, but can also lead to dose-related toxicity. To remove these impurities, accepted purification methods often use multiple chromatographic steps, such as anion exchange, affinity, and size-exclusion. These purification procedures are costly.

U.S. Pat. No. 5,561,064 ('064) demonstrates efforts involved in obtaining pharmaceutical grade plasmid DNA from bacterial cultures. This patent describes a method that uses polyethylene glycol to lyse bacterial cells, followed by a series of chromatographic separations without using additional toxic chemicals, to produce cleaner DNA for gene therapy applications. This method still relies upon producing plasmids in a potentially toxic cell environment, even though no additional components are added in purification.

In contrast, cell free nucleic acid amplification can provide significant cost savings due to streamlined production and simplified purification, and it also eliminates impurities typically associated with bacterially produced plasmids. In vitro amplification, such as polymerase chain reactions (PCR), has been used successfully in the laboratory since mid 1980s. PCR is fast and affordable. However, PCR relies on quick thermal cycling, which is impractical for large scale application.

In vitro isothermal amplification techniques are known, but were primarily used in studying the mechanisms of nucleic acid replication. In 1984, Blanco and Salas isolated phage Phi29 DNA polymerase, which is a highly processive, strand displacement polymerase. Phi29 DNA polymerase can reliably reproduce DNA strands greater than 70 kilo bases long (the full length of Phi29 genome) (Blanco, L. and Salas, M., PNAS 81(17): 5325-5329, 1984). Phi29 polymerase requires a terminal protein, a double-stranded DNA binding protein and a single-stranded DNA binding protein (SSB) for efficient isothermal amplification of DNA in vitro (Blanco, L. et al., PNAS 91: 12198-202, 1994).

U.S. Pat. No. 5,001,050 ('050) claims the use of the Phi29 DNA polymerase in DNA sequencing, DNA amplification, and the synthesis of DNA greater than 10 kilobases long. Related U.S. Pat. Nos. 5,198,543 ('543) and 5,576,204 ('204) claim modified forms of the enzyme, including an exonuclease-deficient form of Phi29 polymerase for improved sequencing reaction. However, these modified polymerases have a lower fidelity and is useful in sequencing reactions only.

In nature, the replication of circular DNA molecules, including plasmid and some viral DNAs, frequently occurs by rolling circle amplification (RCA), whereby the circular DNA template is replicated into a long concatamer of tandem repeats. The concatamer is subsequently cut and packaged into a protein coat (Dean, F. B. et al., Genome Res. 11: 1095-99, 2001). Using RCA, several laboratories have developed methods to amplify DNA for various purposes, including DNA sequencing, cloning, library construction, and screening (Inoue-Nagata, et al., J. Virological Methods 116: 209-211, 2003).

U.S. Pat. Nos. 5,654,033, 6,210,884, 6,316,229, 6,344,329, and 6,797,474 ('033, '884, '229, '329, and '474, respectively) relate to RCA techniques, but only for research applications, such as determining the number of copies of a target message in a given sample. The method first converts DNA samples into circular templates and then uses controlled cycle amplification to determine the relative amounts of the original messages. Another application combines such quantitative amplification with the ability to detect mutant alleles of a target gene.

U.S. Pat. Nos. 6,280,949 and 6,124,120 teach application of RCA in the amplification of an entire genome, using random primers. Some embodiments combine random primers with specific primers to amplify specific regions of the genome.

U.S. Pat. No. 6,329,150 teaches a modified RCA technique using primers designed to nest on newly synthesized DNA to initiate secondary synthesis of already amplified product. In this way, amplification can occur exponentially and provides an isothermal alternative to PCR for the detection of multiple targets simultaneously. U.S. Pat. No. 6,255,082 teaches the use of long terminal repeats to facilitate these nested amplifications. These patents modify RCA to quickly identify the presence of a particular sequence in a sample.

U.S. Pat. No. 6,642,034 teaches a general method of amplification using multiple primers and a strand displacing DNA polymerase. This method is used to identify target sequences, amplification of whole genomes, detection of target sequences or mutations, the synthesis and detection of address tags, and the synthesis of oligonucleotides.

Other applications of RCA include sequencing, cloning, mapping, genotyping, probe generation and diagnostic screening. Published U.S. Patent Application No. 2003/0207267 teaches the use of different DNA polymerases having 3′,5′-exonuclease activity to simultaneously amplify target circles using multiple primers to improve the rate of amplification.

Most of the RCA technology to date has focused on utilizing the Phi29 DNA polymerase even though the use of other polymerases is occasionally addressed. This is because Phi29 polymerase is highly processive, which enables it to synthesize long concatamers of nucleic acid quickly, and has a strand displacement activity, which enables it to continuously synthesize new nucleic acid sequences while displacing any secondary primers it might encounter. In addition, it can produce large amounts of high fidelity nucleic acid in a relatively short period of time without thermal cycling. Phi29 polymerase has an extremely low average error rate of 4×10⁻⁶ (Esteban, J. A., et al., J. Biol. Chem., 268(4): 2719-2726, 1993).

Although most RCA techniques use Phi29 DNA polymerase, U.S. Pat. Nos. 6,576,448 and 6,235,502 disclose the use of bacterial DNA polymerase III (Pol III) in RCA. Pol III reportedly has a clamp-like activity that provides an increased rate of DNA synthesis (about 700-800 nucleotides per second), and it may be optimized by adding helicases or stabilizing proteins. Bacterial DNA polymerase I (Pol I) has also been used in RCA to amplify templates smaller than 100 bp (Fire and Xu, PNAS 92: 4641-45, 1995). Pol I uses predominantly single stranded templates so small circular templates can be readily formed without steric hindrance which is often associated with extremely short double-stranded templates. U.S. Pat. No. 5,614,365 discloses a modified Pol I that includes a sequence of T7 DNA polymerase to increase its efficiency by up to 500-fold. This polymerase has a reduced ability to discriminate between deoxy- and dideoxynucleotides and is good for sequencing reactions.

Typically, an RCA reaction can utilize either a single- or double-stranded nucleic acid template. Phi29 DNA polymerase can use single-stranded or double-stranded templates, while Pol I can only use single-stranded templates. In addition, Phi29 polymerase can use RNA or DNA templates. Therefore, Phi29 polymerase has a more ubiquitous application in RCA and other amplifications.

U.S. Pat. Nos. 6,368,802, 6,096,880 and 5,714,320 teach the use of RCA to produce RNA or DNA oligonucleotides (28-74 nucleotides long), using an appropriate polymerase and a small single-stranded circular DNA template. The DNA oligonucleotides thus produced lack genetic information needed for expression inside a cell.

Moreno S, et al. developed minimalistic, immunogenically defined gene expression vectors (MIDGE) for producing DNA vaccines (Vaccine, 22: 1709-1716, 2004). These vectors contain only the minimal sequence for eukaryotic gene expression and induction of immune responses. In addition, the ends of the linear expression cassettes can be modified to have hairpin loops to increase their longevity and expression efficiency. Although this approach represents a major advance, it involves a labor intensive process because removal of unnecessary bacterial sequences occurs after purification of plasmids from bacterial cultures. The MIDGE process still relies upon bacterial plasmids as precursors.

In a similar manner, Chen, Z. Y. et al. has developed a minicircle which removes bacterial sequences from the vector. They showed that cis-linked bacterial sequences can silence eukaryotic expression (Human Gene Ther. 16: 126-131, 2005) They also showed that linear expression cassettes, lacking any linked bacterial genetic material, express 10-100 times more efficiently than a covalently closed circular plasmid that has an expression cassette linked to a bacterial plasmid backbone. Expression of bacterial free expression cassettes lasts up to 90 days following transfection.

This group describes this process in Published U.S. Patent Application No. 2004-0214329, which teaches circular expression cassettes, therapeutic compositions made from them, and methods of introducing the circular expression cassettes into target cells. In addition, this application uses attB and attP sequences flanking the expression cassette to facilitate removal of bacterial sequence by recombination. Following bacterial culture under normal conditions, the bacteria, which also carry inducible cassettes encoding a recombinase, can be induced to produce the recombinase to induce recombination at the attB and attP sites after sufficient plasmid is produced. The intracellular recombination generates two separate minicircles: one with the expression cassette and the other with bacterial genetic materials. Although streamlined for minimizing bacterial gene contamination, this method still relies upon the production of plasmid in bacterial culture.

Therapeutic applications using DNA require strict adherence to safety and effectiveness standards (Prazeres, D. M., et al., Trends Biotechnol 17(4): 169-173, 1999). Plasmid DNA produced in large-scale facilities should be free of contaminating genomic DNA (<10 ng/dose), host proteins (<10 ng/dose), RNA (non-detectable on 0.8% agarose gel), and endotoxins (<1 Unit/kg body weight, or <0.1 EU/ug plasmid). In addition, the plasmid should be sterile and preferably in supercoiled form that can be more efficiently expressed. Other contaminants that need to be removed from the final preparation include purification reagents, such as ethidium bromide, chloroform, phenol, lysozyme, proteinase K, RNase A, and any potential contaminants that may leach from the purification columns, such as quaternary amines from anion exchangers.

These standards of purity have primarily been addressed using extensive and often expensive purification techniques on bacterial produced plasmid. As stated above, accepted purification methods primarily use multiple chromatographic procedures and may include a combination of anion exchange, affinity, and size-exclusion chromatography purification steps. It is significant that the purification methods needed for the production of therapeutic quality plasmid from bacteria requires specialized equipment, expensive resins, extensive housing facilities and time. Current costs for non-GMP (research quality) plasmid DNA range between $30,000-150,000 per gram of final product, and the costs for GMP quality DNA are approximate 2-3 folds higher. In short, the bio-manufacture of therapeutic DNA using bacterially produced plasmid DNA can be prohibitively expensive.

Methods that focus on utilizing a cell free amplification would provide a significant cost and time savings because of the ability to avoid exposure to a multitude of bacterial contaminants. Everything in the cell free system is clearly defined. Providing that only high quality reagents and enzymes are used, only trace contaminants would be in the system. Thus, final purification can use simple procedures such as dialysis, ultrafiltration and/or gel filtration, and only small volumes of reaction mixture need be purified.

To date, the RCA system has not been adapted the cell free production of nucleic acid for use in gene therapy, DNA vaccines or other therapeutic applications. To the contrary, most of the literature suggests that its usefulness is for strictly diagnostic and research purposes, including sequencing, genomic amplification, genomic analysis, tagging, cloning, PCR-type applications, library construction and other analytical applications.

### BRIEF SUMMARY OF THE INVENTION

One aspect of the invention relates to optimizing the in vitro RCA system to produce a cell free system for large-scale (e.g., >1 mg) nucleic acid production, using streamlined expression cassette templates, highly specific or random primers, high-fidelity polymerases, and a minimalistic buffer system. This system can be used to produce large amounts of nucleic acids, in small volumes, in short periods of time, with the need for only minimal and inexpensive purification procedures. Thus, the system can produce high-quality therapeutic grade nucleic acids for any basic analytical or research purpose, but more importantly for therapeutic use.

The current invention combines several techniques for the purpose of affordably producing large amounts of high-quality nucleic acid for therapeutic, diagnostic and research applications. The method of the invention can produce 250-300 times more nucleic acids than what is produced in a comparable volume of bacteria culture. In the current invention, there is no contaminating source of endotoxin other than what is minimally contained in the reagents used. Additional advantages include: the capability of producing large fermentation-like quantities of product in a small laboratory flask; the requirement of only a minimal number of reagents; ability to produce large amounts of product in a relatively short period of time; and streamlined purification procedures. Together, these advantages translate into an affordable way to produce large quantities of high-quality nucleic acids for therapeutic use.

In one embodiment of the invention, a modified plasmid lacking typical genetic sequences needed for plasmid selection and replication in bacteria is used as a template. Any circular nucleic acid template can be used. In accordance with embodiments of the invention, a template may be a circular expression cassette containing a gene of interest flanked by genetic elements needed for expression and processing of the expressed product in a host.

Although regular plasmids can be replicated using a method of the invention, streamlined templates having no extra genetic sequences offer multiple benefits: it eliminates any extraneous sequence that may silence the expression of the sequence of interest; the smaller construct is more compact and can be more efficiently taken up by the target cell, leading to higher transfection efficiency; and it is more cost effective due to production of a larger quantity of an expression cassette with less material, a statistical increase in fidelity of the final product and no need for extensive purification.

Although random primers or sequence-specific primers can be used, sequence-specific primers are more efficient and economical in large scale amplifications. Primer sizes may range from four to greater than twenty nucleotides, and they may comprise modified bases and/or backbones for increased affinity, stability and prolonged storage. In one embodiment, a specific primer with phosphorothioate end-modification may be used to produce a large amount (about 1.5 mg in 1 ml) of nucleic acid.

The amplification step can use any specific polymerase providing buffer and temperature conditions are adjusted to accommodate the specific needs of that polymerase. An example would be to include the necessary denaturation and annealing steps when using a high temperature taq-like polymerase. However, preferred embodiments of the invention use processive, strand-displacing polymerases, such as Phi29-like polymerases, to efficiently amplify templates without thermal cycling. Preferred embodiments use Phi29 or Phi29-like polymerases, but other polymerases such as Pol I and Pol III, T7 DNA polymerase, and their derivatives can also be used. The invention can also use other modified or chimeric polymerase designed to improve efficiency and/or fidelity.

Following amplification, the nucleic acid product may be further processed in a manner to facilitate its intended use. Research purposes, including detection, identification or sequencing, would typically only require shorter linear units (delivery unit) of the concatamer which may be attained by either restriction enzyme digestion or by physical or chemical methods such as shearing or induced cleavage at specific, photolabile nucleotide. Cellular transfections may be accomplished with a variety of forms, but higher efficiencies of uptake are typically attained with circular or supercoiled nucleic acid. One embodiment incorporates a subsequent ligation step using DNA ligase to make circular nucleic acids (CNAs). Another embodiment uses a recombinase or a similar enzyme to circularize the delivery unit into CNAs. Another embodiment includes an additional step using DNA gyrase to supercoil the circular product to produce supercoiled CNA (sCNA).

If the product is intended for expression in eukaryotic cells, uptake by the cell is critical, whether in culture or in therapeutic applications. Transfections can be accomplished using circular, supercoiled CNA or specially designed linear forms which may be stabilized with modifications in the internal base and/or the ends of the linear unit. Such modifications include: blunting the ends by filling in with a Klenow fragment-like enzyme; phosphorothioating the ends of linear strands with appropriately modified bases; incorporating other modified bases either during the amplification process or following digestion of the concatamer, which stabilize or minimize degradation of the linear in vivo; and designing the expression cassette to comprise stabilizing sequences which facilitate rapid uptake and/or prolong longevity of expression of the cassette once inside the cell (Kay, M. A. et al., Molec. Ther. 3(3): 403-410, March 2001).

The degree of modification or processing following the cell free amplification step is dependent upon the intended use for the product. The final processed product is then purified in order to eliminate reagents, contaminants, and/or any alternative forms of the product. Different forms of the product may include linear fragments, open circles, covalently closed circles comprising monomers, dimers, trimers, etc., as well as supercoiled circles. The intended form is dependent upon the specific application and may alternate between any of the aforementioned forms. Depending upon the number of reagents used and on the degree of purity needed, the product can be subjected to chromatography, ultra filtration, dialysis, nucleic acid precipitation, or any other appropriate method known in the field. Those embodiments incorporating gel filtration and/or dialysis can provide high quality products for therapeutic applications.

### BRIEF DESCRIPTION OF THE DRAWINGS

FIG. 1 shows multiple mechanisms for generating useful templates. For example, templates may be produced by plasmid modification, PCR amplification, chemical synthesis, or cDNA synthesis.

FIG. 2 shows an RCA-based amplification process according to one embodiment of the invention. The process uses a polymerase to synthesize a concatamer from the circular template. The concatamer may be processed into smaller fragments, which may comprise at least one intact expression cassette. The synthesized product may be used as short linear units, circularized nucleic acids (CNAs), or supercoiled circular nucleic acids (sCNAs).

FIG. 3 shows a method according to another embodiment of the invention. The method involves separately amplifying the forward (A) and reverse (B) strands of a double-stranded template. In two separate reaction vessels, each strand is amplified and circularized into single-stranded circles. In each vessel only one strand is amplified using a strand-specific primer. A second oligonucleotide comprising a sequence for a restriction site (OR1 or OR2) is then annealed to a pre-designed site in the single-stranded concatamer, whereby short segments of double-stranded templates are generated to enable digestion by a restriction enzyme. Following digestion but prior to denaturation, the double-stranded ends are circularized using a DNA ligase. Following ligation, the oligonucleotide is denatured to from single-stranded circles, which are then combined with the complementary single-stranded circles to form double-stranded circles that comprise only monomers. This method minimizes the formation of dimers, trimers and other multimer byproducts.

FIG. 4 depicts the scale-up of the RCA process according to one embodiment of the invention. The process involves sequential addition of template, primer, buffer components and enzymes at the designated times and shifting to the designated temperatures. This provides an efficient method for producing large amounts of product in a short period of time. Diluting the reaction volume prior to ligation favors the formation of monomeric circular product.

FIG. 5 depicts the design for an automated amplification apparatus. (A) represents a model where large numbers of individual reactions comprising volumes of less than 1 ml can be used to amplify numerous individual templates simultaneously. (B) shows the use of a single vessel enabling the synthesis of large quantities of a single DNA product. (1) Programmable computer access to control reaction parameters; (2) monitor for evaluating and adjusting reaction parameters; (3) temperature controlled chamber for stock solutions including enzymes, buffers, and other components; (4) dispensing port for addition of reagents to the reaction vessels; (5) temperature controlled reaction vessel; (6) multiple-well dispenser; (7) multiple-well reaction vessel plates; (8) temperature controlled chamber for multiple-well plates.

FIG. 6 schematically summarizes various mixing strategies for viscous reaction mixtures: (A) propeller-like mixing vessel; (B) perforated disk mixing vessel; (C) recycling mixing vessel using a peristaltic pump. In (C), (1) is an adjustable automated control and port for calibrated addition of reagents held in (2). The adjustable control (1) enables controlled mixing of reagent with a small stream of reaction mixture and supports the overall mixing of the reaction mixture by depositing the reagent modified reaction mixture back into the chamber at a position opposite the outlet port. Continued pumping without reagent facilitates thorough mixing.

FIG. 7 depicts a process for intra-molecular ligation. Following amplification and digestion of DNA in vessel (B), the reaction mixture is added slowly to a second vessel (A) containing a ligation cocktail. Slow addition of the DNA into vessel (A) provides sufficient dilution of the DNA to facilitate monomeric CNA formation.

FIG. 8 shows results of IgG antibody titers against gp160 produced in Balb/c mice after immunization with a plasmid, a short expression cassette (synthetic DNA, synDNA) produced in accordance with one embodiment of the invention, and a control solution. These results clearly show that the synthetic DNA is effective in inducing immune responses in mice.

FIG. 9 shows results from immunization of rabbits using a plasmid or a synthetic DNA (expression cassette), prepared in accordance with one embodiment of the invention, containing a sequence for the Hepatitis B virus small surface antigen (HBs(S)). These results clearly show that the synthetic expression cassette of the invention is effective in inducing immune responses in rabbits.

FIG. 10 shows immunization results following the injection of BALBc mice against influenza H1N1 virus. The figure shows virus-neutralization titers recorded as the last dilution in which virus replication was inhibited for the various genetic immunization experiments.

### DETAILED DESCRIPTION OF THE INVENTION

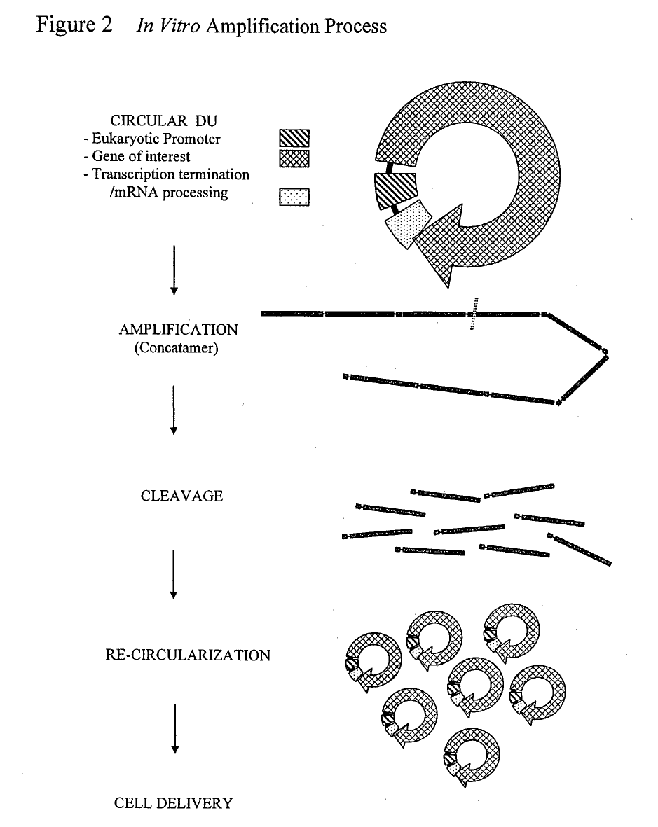

Embodiments of the invention relate to methods and apparatus for producing large amounts of high-quality nucleic acids. Methods of the invention use rolling circle amplification (RCA)-based cell-free systems to produce therapeutically useful, minimally contaminated nucleic acid products (FIG. 2).

“Nucleic acid,” “oligo,” or “oligonucleotide,” as used in the context of this invention, may be DNA or RNA, or its analog (e.g., phosphorothioate analog). Nucleic acids or oligonucleotides may also include modified bases, backbones, and/or ends. Synthetic backbones may include phosphorothioate (Pt), peptide nucleic acid (PNA), locked nucleic acid (LNA), xylose nucleic acid (XNA), or analogs thereof that confer stability and/or other advantages to the nucleic acids.

As shown in FIG. 2, a method in accordance with one embodiment of the invention is based on RCA. The process uses a polymerase to synthesize a concatamer from the circular template. The concatamer may be processed into smaller fragments, which may comprise at least one intact expression cassette. The synthesized product may be used as short linear units or further processed to produce circularized nucleic acids (CNAs) or supercoiled circular nucleic acids (sCNAs).

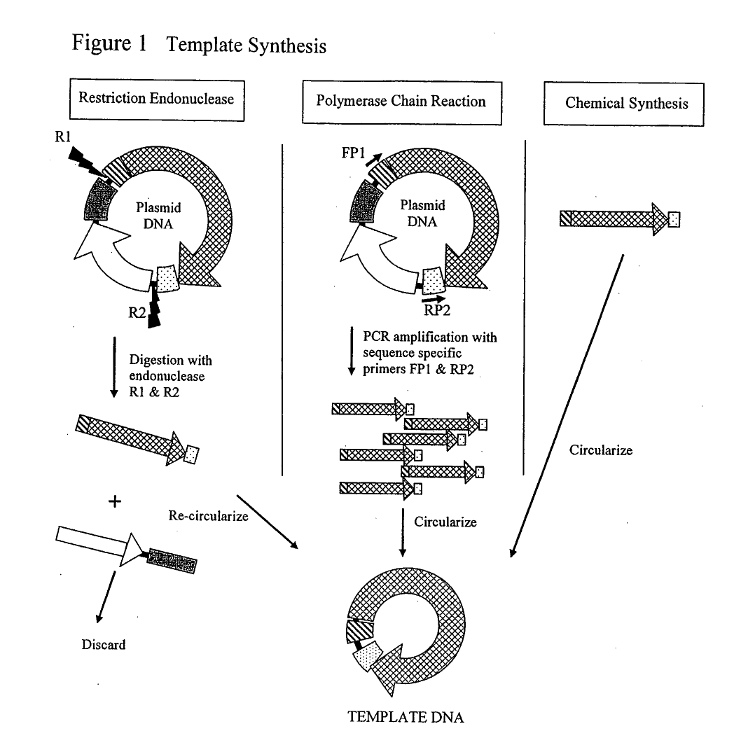

Methods of the invention may start with DNA or RNA templates. Those starting with RNA templates would include a reverse transcriptase, such as the avian myeloblastosis virus reverse transcriptase, to make a cDNA template. Any method known in the art may be used to prepare a circular template for use in a method of the invention, as shown in FIG. 2. Some of these methods will be described in detail later with reference to FIG. 1.

Single-stranded binding proteins can be used to stabilize the templates and improve efficiencies of the amplifications for some polymerases. Additional enzymes can also be included in the amplification reaction to repair mistakes. Protein mediated error correction enzymes, such as the mutation splicing protein (MutS), can also effectively improve a polymerase's overall fidelity and may be used either during or after the amplification reaction (Carr, P., et al., Nuc Ac Res 32(20): e162, 2004).

Depending upon the intended use, the DNA polymerases used in a method of the invention may be any known prokaryotic, fungal, viral, bacteriophage, plant or eukaryotic DNA polymerases and may include holoenzymes and any functional portions of the holoenzymes or any modified polymerase that can effectuate the synthesis of a nucleic acid molecule. Useful DNA polymerases include: bacteriophage phi29 DNA polymerase, other phi29-like polymerase (such as phage M2 DNA polymerase, phage B103 DNA polymerase, or phage GA-1 DNA polymerase), phage phi-PRD1 polymerase, VENT DNA polymerase, DEEP VENT DNA polymerase, KlenTaq DNA polymerase, DNA polymerase 1, Klenow fragment of DNA polymerase I, DNA polymerase III holoenzyme, T5 DNA polymerase, T4 DNA polymerase holoenzyme, T7 DNA polymerase, Bst polymerase, rBST DNA polymerase, N29 DNA polymerase, TopoTaq DNA polymerase, and ThermoPhi™ DNA polymerase. Preferred embodiments of the invention use Phi29 polymerase, Phi29-like polymerase, or other high-fidelity polymerases (e.g., hybrid fusion polymerase).

Preferred embodiments of the invention use processive, strand-displacing polymerase to amplify DNA under conditions for high fidelity base incorporation. In the context of this invention, a high fidelity “DNA polymerase” is one that under recommended conditions, has an error incorporation rate equal to or lower than those (1.5×10⁻⁵-5.7×10⁻⁵) associated with commonly used thermostable PCR polymerases, such as Vent DNA Polymerase, KlenTaq DNA Polymerase, or T7 DNA Polymerase. Additional enzymes may be included in the reaction to minimize misincorporation events including protein mediated error correction enzymes, such as MutS, which effectively improves polymerase fidelity either during or following the polymerase reaction (Carr, P. et al, Nuc Ac Res 32(20):e162, 2004).

Similarly, a high fidelity “RNA polymerase” has an error incorporation rate equal to or lower than those of common RNA polymerases (Promega Technical Information). RNA polymerases useful in this invention include T7 RNA polymerase, SP6 RNA polymerase, T3 RNA polymerase, and their modified or chimeric versions.

During the amplification reaction, the circular template is replicated by a polymerase in the presence of deoxyribonucleoside triphosphates (dNTPs), ribonucleoside triphosphates (NTPs), or modified counterparts, forming a long concatamer comprising tandem repeats of the template. The concatamers are subsequently cleaved, e.g., by restriction enzyme cleavage or physical shearing, into smaller fragments referred to as “short expression cassettes” (SECs). An SEC contains a sequence of interest and may optionally contain eukaryotic expression sequences (or cassettes). Preferred embodiments use SECs that comprise at least one eukaryotic expression cassette. Unlike conventional, bacterially produced plasmids, an SEC of the invention consists solely of a sequence of interest flanked by the intended eukaryotic sequences, but no bacterial genetic material.

The “short expression cassette” may include: an eukaryotic promoter recognized by the targeted cell; the sequence of interest which may be an intact gene, a gene fragment, or a specific sequence of interest (SOI); and a transcription termination sequence. The short expression cassette may be flanked by additional sequences to facilitate ligation (e.g., making CAN) or to stabilize a linear fragment. The expression cassette, together with the desired flanking sequences, comprises a “delivery unit” (DU), and does not contain unnecessary genetic material which is solely used for selection and replication of a plasmid produced in bacterial culture. By minimizing bacterial genetic material that has no value inside a eukaryotic cell, it is possible to generate high concentrations of high quality, bio-active DNA molecules. The nucleic acid produced is smaller than a typical plasmid, is more efficiently transfected into a cell, and upon transfection, is more efficiently expressed inside the cell.

Enzymatic or chemical methods can be used to improve the homogeneity of the final products by eliminating DU with mismatched nucleotides resulting from errors in polymerization. For example, enzymes used in mutation detection (such as resolvases, T4 Endonuclease VII, or T7 Endonuclease I) or other enzymes used to detect gene mutations or polymorphism and in high-throughput screening of point mutations (such as TILLING) may be used to accomplish this goal.

As noted above, any method may be used to prepare a circular template for use with methods of the invention. FIG. 1 shows three commonly used methods for generating useful circular templates that include at least one sequence of interest (SEC or DU). One method involves enzymatic modification of an existing plasmid, whereby the DU including the eukaryotic expression cassette is selectively excised from a plasmid by restriction endonuclease digestion. The DU is free of the origin of replication or selectable marker genes, such as an antibiotic resistance mediator, which can silence expression of the SOI in vivo.

A preferred embodiment of the invention uses a template comprising an intact eukaryotic expression cassette with flanking sequences on either side of the cassette (FIG. 1) to enable circularization of the linear SEC into a CNA. The template can be any single- or double-stranded nucleic acid (DNA or RNA), which is converted into a circular template and includes plasmid as well as minicircle DNA. Pre-ligation reactions may be carried out as in the case of using padlock probes (Baner, J., et al., Nuc Ac Res 26(22): 5073-78, 1998).

Double-stranded templates may need to be denatured initially to optimize the polymerase reaction depending upon the polymerase used. In such reactions, both the forward and reverse strands can be simultaneously amplified in the same reaction. Subsequent processing may then require the addition of a restriction endonuclease, a ligase, and/or a gyrase. The products may then be purified to yield DUs for therapeutic applications.

A second method for making the templates involves PCR amplification from a larger DNA template using specified oligonucleotides that flank the specific expression cassette to produce relatively short DUs for circularization.

A third method shown in FIG. 1 involves chemical synthesis of oligonucleotides (oligos) to make a single nucleic acid strand or complementary strands that are then circularized to produce a template containing a DU or expression cassette.

During amplification, the template may be freely suspended in solution or bound to a support, such as a chromosome or protein (U.S. Pat. No. 5,854,033), or a solid support such as glass or polystyrene beads.

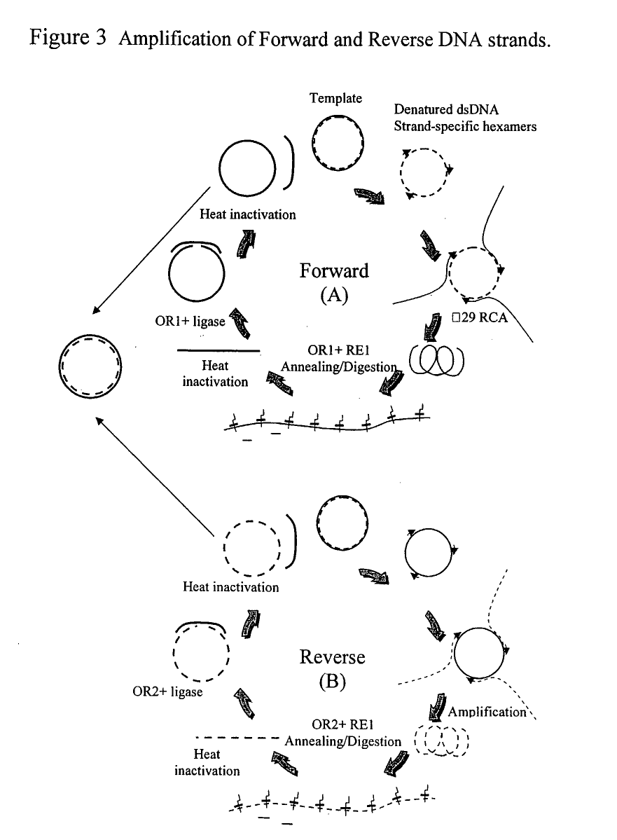

An alternative method in accordance with embodiments of the invention is shown in FIG. 3. As shown, each strand of a double-stranded template may be separately amplified using appropriately designed primers to produce single stranded concatamers of DUs. The separately amplified concatamers are individually mixed with oligos containing specific restriction sites and cleaved with the restriction enzymes. The temporarily double-stranded ends of these fragments are ligated to form circular single-stranded products (Dahl, F., et al., PNAS 101(13): 4548-53, 2004). The advantage of this method is that the single-stranded circles of each reaction can then be combined to form a single class of double-stranded monomeric circles, thus avoiding the need to purify the monomers away from other multimeric forms of the reaction. The monomeric circles can then be supercoiled with a DNA-gyrase or a similar enzyme to improve the efficiency of uptake and expression of the expression cassette.

Multiple embodiments use a circular, double-stranded DNA template with primers that specifically bind at designated sites to initiate concatamer synthesis. The primers can comprise any of the different variations of “nucleic acid” to improve stability, and may be of various lengths where the length is determined by the annealing temperatures of the DNA polymerase used. The primer sequences may comprise random or specific sequences, may be designed to have specific sequence alterations, or may include tags or detection sequences that are non-complementary to the template in order to facilitate manipulation or analysis of the amplified sequences. For example, in one embodiment, random hexamers are used to effectively amplify a DU, which upon processing and transfection into cells, would produce the desired effects. Other embodiments use specifically designed primers which enable the RCA reaction to be controlled by spacing out the initiation sites and by using primers of controlled affinity for optimizing the amplification reaction conditions. Sequence-specific primers, as short as a tetramer, may be used to effectively amplify a specific DU.

In most applications, the polymerases, restriction endonucleases, ligases, and other enzymes as used in this invention constitute soluble forms of the enzymes. However, solid phase amplification reactions or solid phase processing reactions including restriction digestion, ligation and supercoiling reactions may also be employed to streamline the amplification process. In addition, fusion proteins comprising optimal regions of different enzymes (especially polymerases) which are designed to improve fidelity, efficiency, and processing or the final product may be used. Recombinant forms of the enzymes containing one or more affinity tags (such as 6×His, S-Tag, Calmodulin-binding peptide, Protein A and others) expressed in bacteria, fungus, plants, insects, or animal cells may also be used. The advantage of using tagged enzymes is that they can be readily eliminated from the final product using affinity chromatography. Following purification, the recovered enzymes, immobilized on a solid matrix through the tag moiety, may be used in subsequent enzymatic reactions.

Following amplification, the concatamer is cleaved into short expression cassettes (SECs) comprising at least one DU, where a single SEC may comprise multiple copies of a DU and may be designed as such in order to optimize delivery and expression. The linear SECs may be directly administered as the linear fragments, circularized fragments (CNA), or supercoiled circularized fragments (sCNA) to facilitate uptake by the target cell. As such, the post-amplification processing would vary according to the intended use.

Processing of the SEC can include any one or more of the following: additional cutting of the SEC with other physical or enzymatic methods; filling in or processing the ends of the SEC either by enzymatic cleavage, as with Klenow, or by chemical methods; internally ligating the two ends of the SEC to produce a circularized CNA; supercoiling the CNA with gyrase-type enzymes including topoisomerase type II; enzymatically or chemically treating any of the forms to have modified internal bases or modified ends; ligating two or more SECs together; or ligating an SEC to a specific ligand to produce a functional conjugate. The term ligand as defined in the context of this invention includes: a nucleic acid, including DNA, RNA, PNA, LNA or modifications thereof; peptides, either to facilitate targeting and cellular uptake or to increase therapeutic efficacy; polypeptides that may be enzymatically active and/or physically functional; aptamers, nucleic acids that recognize, bind and modify a protein's function; bio-physical tags, including fluorescent, magnetic, and radiolabeled components; as well as polymers which facilitate either stabilization of the nucleic acid, or targeting of the product to the intended cell or tissue.

Therapeutic applications that can be successfully administered using DNA produced by the invention include several approaches to DNA therapy, including antibody production and gene silencing. For example, antibodies can be produced in vivo following successful administration of appropriate expression cassettes designed to prevent or treat a disease caused by a pathogen, such as influenza and HIV viruses. For example, the sequence encoding the influenza haemagglutinin protein under the control of an eukaryotic promoter may be used to elicit a humoral and cellular immune response in animals targeted by influenza A virus. Similarly, the expression of a sequence encoding a truncated Human Immunodeficiency Virus (HIV) envelope protein can successfully induce an effective immunogenic response against HIV in mice.

The amplified nucleic acid of this invention can also be shown to mediate targeted gene silencing in vivo. Herpes Simplex Virus (HSV), which causes painful blisters and sores on various parts of the body, and Herpes Zoster, which causes chicken pox (initial infection) and shingles (upon recurrence), are members of the same family of viruses which require the expression of both ICP4 and ICP47 to effectuate a viral infection. Upon transfection in cell culture, amplified SECs expressing antisense oligos specific for ICP4 or ICP47 may be used to modulate these protein expression in vivo and can minimize further proliferation of the virus. Expression of an anti-ICP4 transcript in vivo successfully silences the ICP4 gene and blocks the production of ICP4 protein in the cell. A similar effect can be seen following expression of the ICP47 expression cassettes produced by this method.

ICP47 functions to inhibit the major histocompatibility complex (MHC) presentation pathway, which is critical for shielding the virus from host immunogenic attack. The gene product of ICP47 binds to a transporter protein involved in the presentation of antigens on the outside of an infected cell, thus blocking the major histocompatibility complex (MHC) class I antigen presentation pathway. Consequently, the HSV-infected cells are masked from immune recognition by cytotoxic T-lymphocytes. Thus, ICP47 plays an essential role in HSV-infection.

Transfecting the lung cancer cell line, A549, with an ICP47 SEC amplified according to this invention can effectively express antisense sequences and block production of the ICP47 protein as assayed by Western blot analysis. There are additional infected cell proteins (ICP's) in the herpes simplex genome that can be similarly silenced.

Other gene silencing targets include the respiratory viruses such as the rhinoviruses, coronavirus, adenovirus, influenza and para-influenza viruses, which are frequently associated with both upper and lower respiratory tract infections including the common cold, pneumonia, asthma, and chronic obstructive pulmonary disease (COPD). The human rhinovirus (HRV) has a single-stranded RNA genome that is approximately 7.2 kb in size with a single-open-reading frame that encodes for a capsid coat protein, an RNA polymerase and two viral proteases. Upon infection, the viral proteins effectively redirect the host machinery to manufacture thousands of viral particles which are eventually released when the cell lyses.

Most rhinoviruses make use of intercellular adhesion molecule I (ICAM-1) as a receptor to infect the cell. Expression of an amplified SEC encoding an antisense to the ICAM-1 message can effectively block expression of the ICAM-1 protein in vivo and may prove to be useful in minimizing viral infection. Other useful strategies for combating respiratory diseases include in vivo expression of antisense-like molecules (antisense, aptamers, triplex forming molecules, and similar molecules) to block activities of essential proteins that mediate infection, such as viral proteases that are required to process viral particles. Other approaches may include using the SECs to block mediators (e.g., bradykinin, prostaglandins, tachykinins, histamine, and various cytokines) of pathogen-induced tissue responses, or to block the cellular receptors that effectuate the physiological effect caused by these mediators.

Other targets for therapeutic applications of this invention include modulating infections caused by the human papilloma viruses (HPVs) which initially manifest infections as benign, non-cancerous warts but in some cases can progress into malignant e growths. For example, genital HPVs can be passed from one person to another through sexual intercourse as well as through oral or anal sex. Virus-infected cervical cells can transition from an initial benign wart, into premalignant cells and eventually develop into a carcinoma. Cervical cancer is probably one of the best known examples of how infection with a virus can lead to cancer. In humans and animals, cell division is primarily regulated by Rb and p53. The E6 and E7 proteins of HPV can attach directly to Rb and/or p53, inhibit the tumor suppressor effects of the proteins and cause the infected cells to reproduce without control (Didelot, C. et al., Intl J Oncology 23:81-87, 2003). While the virus serves only as the initiating event, over time some of the wildly growing cells develop permanent changes in their genetic structure that cannot be repaired. By expressing antisense-like constructs designed to block E6 and E7, viral infections would be rendered ineffective.

Other types of HPV infections may manifest themselves as warts on or around the genitals and anus of both men and women and are also valid candidates for therapeutic antisense-like expression using the nucleic acid produced by this invention. In women, visible warts may also appear in the cervix. This type of a genital wart is known technically as Condyloma acuminatum and is generally associated with two HPV types, numbers 6 and 11. These warts rarely develop into cancer, and are considered to be “low-risk” viruses. Other sexually transmitted HPVs have been linked with genital or anal cancers in both men and women. These are called “high risk” HPV types and include HPV-16, HPV-18, HPV-31, HPV-45, as well as some others. High risk HPV types aren't usually contained in visible warts, but both high-risk and low-risk HPVs can cause the growth of abnormal cells in the cervix. Both types of HPV infections can be effectively controlled with an effective in vivo antisense-like expression therapeutic.

The amplification reaction of the invention can also be used to amplify either an intact plasmid comprising bacterial sequences, or a modified version of the plasmid to exclude these sequences. For example, a single-stranded DNA expression vector, pssXE, which includes: 1) a Mouse Moloney leukemia viral reverse transcriptase (MoMuLV RT) gene coding for a truncated but fully active RT; 2) a primer binding site (PBS) with flanking regions essential for reverse transcription initiation by MoMuLV RT; 3) a target gene coding sequence for the production of an antisense, an aptamer, a DNA enzyme, or a sequence that induces triplex formation; and 4) a stem-loop structure designed for the termination of the reverse transcription reaction, as an intact expression cassette, can be effectively amplified according to the invention. The amplified products can be transfected and used to effectively silence mammalian, viral, and bacterial genes. Upon expression inside the cell, the transfected RT subsequently uses an endogenous host tRNA (e.g., tRNAPro or tRNAVal) as a primer to bind to a primer binding site (PBS) at the 3′ end of the RNA transcript and initiates ssDNA synthesis. After reverse transcription, ssDNA may be released when the mRNA template is degraded by RNase H or the RNase H activity of RT.

Delivery of the nucleic acid (SEC) can be accomplished by simple injection of a naked nucleic acid in stabilizing buffer into the targeted recipient. Embodiments of the invention may also use delivery vectors which help target and delivery of the nucleic acid into the cell (Dias, N. Molec Cancer Ther 1: 347-355, 2002). Some embodiments use a viral vector system which may be an attenuated virus system, a viral packaging system that includes few or no immunogenic protein (Srivastava, I. K. and Liu, M. A. Ann Intern Med. 138: 550-559, 2003). Other embodiments include the use of neutral or cationic liposomes which either encapsulate the nucleic acids or bind the nucleic acid by electrostatic interactions. These embodiments may also use helper molecules (e.g., chloroquine or 1,2-dioleoyl-sn-glycero-3-phosphatidylethanolamine) to prevent sequestering of the delivered nucleic acid in the endosomal compartments. Some of the commercially available liposomal vectors include Lipofectin, Eufectins, Cytofectin and Lipofectamine.

Other methods of delivery include covalent coupling of the nucleic acids to cationic peptides, which may modulate the permeability of plasma membrane by physical interactions, receptor- or transporter-mediated mechanisms. Such coupling increases the effectiveness of the delivered nucleic acid which is delivered directly into the cytoplasm and is readily transported to the nucleus for expression (Luo, D. and Saltzman, W. M. Nature Biotech 18: 33-37, 2000). Still other embodiments use cationic polymers which interact electrostatically with the therapeutic nucleic acid to deliver nucleic acid to the cell. Cationic polymers, for example, include poly-L-lysine (PLL), polyethylene glycol (PEG), PEG-block-PLL-dendrimers, polyamidoamine (PAMAM) dendrimers, polyalkylcyano-acrylate nanoparticles, and polyethyl-eneimine (PEI) and its conjugates (such as mannose-PEI, transferin-PEI, linear PEI).

Aerosol delivery is a noninvasive mode of delivery to airway epithelium and pulmonary surfaces. For example, formulations comprising PEI and nucleic acid can effectuate high level airway or pulmonary transfection upon delivery by nebulization. This application of PEI-nucleic acid complexes can effectuate higher levels of gene expression than many cationic lipid formulations, and exhibits a remarkably high efficiency (nearly 100%) of transfection into cells of the airway epithelium and lung parenchyma. In addition, repeated aerosol administrations of PEI-based formulations are associated with very low toxicity. This delivery method only minimally induces expression of tumor necrosis factor alpha (TNF-α) and interleukin 1 beta (IL-1β) as compared to intravenous injections of PEI-nucleic acid or aerosol delivery of cationic liposome-nucleic acid complexes.

A frequent problem of using bacterially produced plasmid DNA results from exposure of the host to unmethylated motifs inherent in bacterially processed DNA. Unmethylated DNA can induce a CpG-mediated cytokine response and the induction of pro-inflammatory cytokines which is a serious problem associated with lung toxicity and reduced efficiency of therapeutic applications. Consequently, the use of bacterially produced DNA has severely hampered many of the current gene therapy approaches used to date. Masking of the CpG response by PEI can facilitate the sustained expression of genes that are delivered via PEI-gene aerosol and, thus, the sustained therapeutic response achieved. When used in combination with the nucleic acid produced by the cell free amplification method of this invention, PEI-based aerosols can be extremely effective delivery systems for DNA therapeutics to lung and airway epithelium.

Some of the embodiments also use long-term release systems. Biocompatible controlled-release polymers such as poly(D,L-lactide-co-glycolide) (PLGA) microspheres and poly(ethylene-co-vinyl acetate (EVAc) matrices can effectuate a controlled, adjustable and predictable release of the bioactive nucleic acid for up to several months, and both components have been approved for therapeutic use by the U.S. Food and Drug Administration.

Physical delivery systems may also be used. Electroporation may be efficient for transferring therapeutics to skin cells, corneal endothelium and other tissues including muscle. Pressure-mediated or hydrodynamic injection can effectuate up to 50% efficiency in mammalian systems. Other methods include ultrasonic nebulization for delivery of DNA-lipid complexes in many different types of cells, including plants, and particle bombardment is also useful for plants.

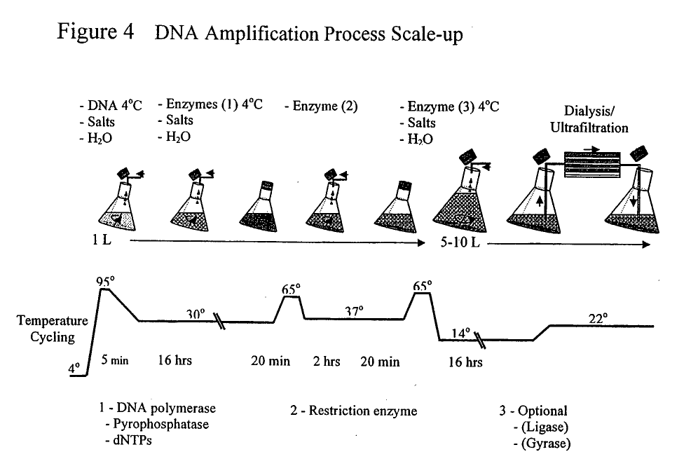

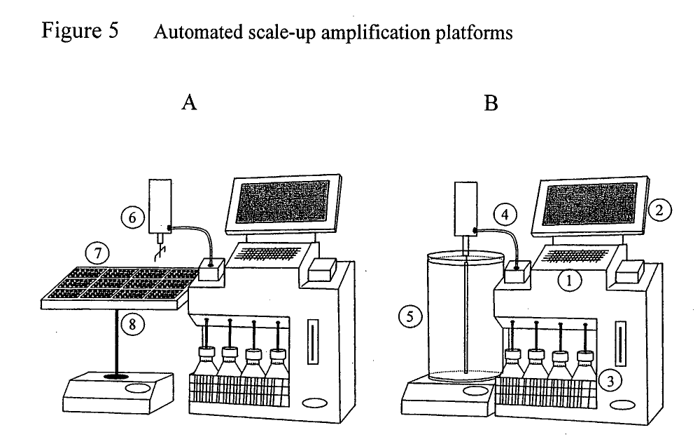

Scale-up of the cell free amplification process may be performed using a semi- or fully-automated platform, where sequential additions of salts, enzymes and nucleic acids, together with temperature and incubation times, can be tightly controlled for optimal efficiency (FIG. 4). In one embodiment, scale-up can be accomplished by increasing the number of reactions while keeping each reaction volume relatively small (<1 ml) whereby the template(s) can be amplified simultaneously using multi-well plates in standard or custom built platforms (FIG. 5A). Alternatively, scale-up may involve larger volumes (e.g., 10 liters) to generate large quantities (kg amounts) of a single nucleic acid product in a single run using a fermenter-like vessel under environmental controls (FIG. 5B). Larger volumes may be used to produce larger yields of product. Multiple platforms of mixed capacities can be arranged in parallel within a confined space and can function in a coordinate manner as part of a larger bio-manufacturing facility that can meet various amplification scale requirements.

The production of large amounts of nucleic acid in a small volume presents the problem of mixing reagents into a highly viscous reaction mixture. The invention includes a reaction vessel that can be either a hardened pre-formed container or a flexible container such as a self contained plastic bag. In the preferred embodiments, the reaction vessel and all components that come in contact with the reaction mixture are clean, sterile and free of any contaminating nucleic acid sequences. The hardened pre-formed container contents are preferably mixed by a device that is contained inside the reaction vessel, but may involve a re-circulating device. The flexible vessel is preferably mixed by a re-circulating mechanism which could include the use of a peristaltic-like pump, or may incorporate an external mechanical device such as an automated squeezing apparatus or a low-energy pulsation device that avoids shearing of the nucleic acid product.

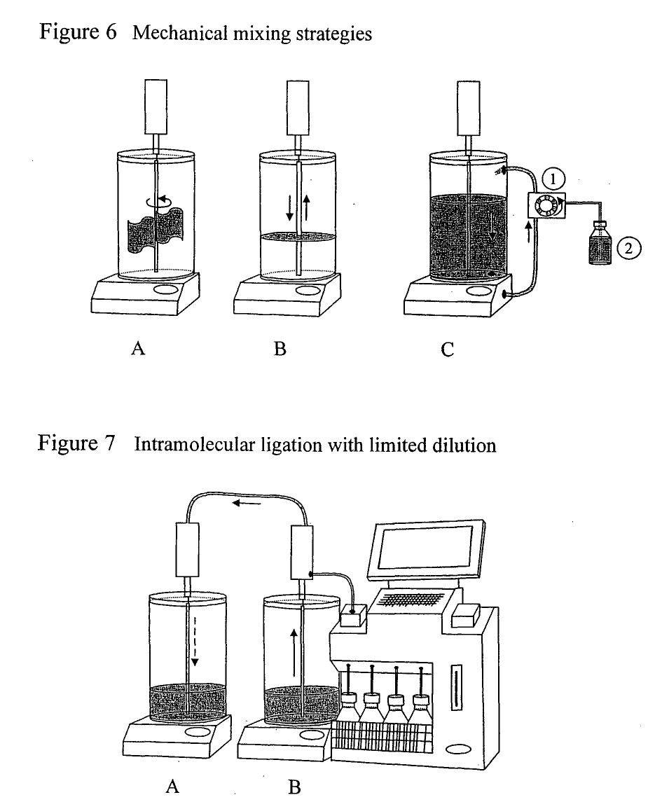

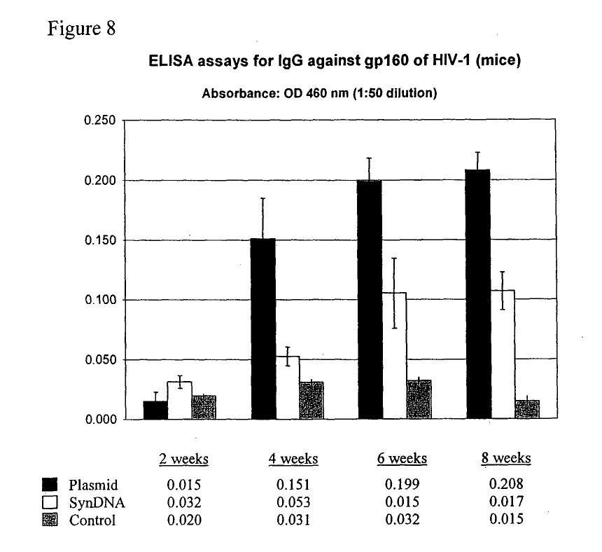

Internal devices can use several different mechanisms including propeller-like stirring devices with electronically controlled speeds and automated timing (FIG. 6A), or controlled liquid displacement processes using a perforated disk fixed to a shaft running from top to bottom within the reaction vessel's inner diameter (FIG. 6B). The disks are raised and lowered at various speeds within the liquid to provide adequate mixing of the reaction mixture. Both of these mixing chambers can be equipped with a dispensing device which may comprise a small tube attached to the shaft of each mixer which delivers various stock components, which are chambered separately outside the mixing vessel, into the reaction mixture using a peristaltic pump to control the precise and sequential delivery of the various reagents.

Another embodiment implements a system where a steady constant flow of the reaction mixture is pumped from and then back into the chamber. For example, an outlet located at the bottom of the chamber enables a small stream of fluid to be combined with an added reagent and then channeled back through an entry port located at the top of the same reaction chamber to effectuate mixing (FIG. 6C). Peristaltic pumps and intake valves control and monitor the dispensing of various solutes and enzymes during the recycling process (FIG. 6C).

Yet another embodiment utilizes the thixotropic nature of the DNA mixture, wherein the mixture is cylindrically configured into an elongated form. Thixotropic compounds can change viscosity according to the degree of shear force applied to the compound. Typically, an increase in the shear force can decrease a thixotropic compound's viscosity. Once the shear force is removed, such a compound will begin to regress to its original viscosity. In this embodiment, the container holding the viscous reaction mixture has evenly spaced pores through which necessary chemicals are injected for processing. Elongation of the viscous reaction mixture through the small diameter cylinders therefore changes the viscosity sufficiently to promote localized mixing with reagents which are slowly infused into the small diameter cylinders and into the less viscous reaction mixture for a sufficiently long period in which to effectuate mixing.

The apparatus preferably includes one or more inline real-time monitoring of all relevant physical and biochemical parameters to verify product stability and maintain quality control and quality assurance, which are necessary to maintain certified good manufacturing practice (cGMP) required for a product acceptable for therapeutic applications. This may include a computer or similar means for monitoring viscosity, nucleic acid concentration, solution turbidity; conductivity; pH; temperature; protein content; endotoxin, bioburden, and/or chemical contaminants arising from degradable components of the system.

Processing of the linear SEC into a circular form requires that the ligation step favor an intramolecular (self-adhering) reaction over an intermolecular reaction. Traditional dilution of the final amplification product can be used to manipulate the molar ratio to favor intramolecular ligation. Preferred embodiments, however, minimize the overall reaction volume by mixing small amounts of the reaction mixture into a ligation cocktail containing the enzyme and buffer components. In one embodiment, the amplified product is added into a small stream of reaction mixture as shown in FIG. 6C, using very slow or pulsating pump rates. Other embodiments dispense the amplified reaction mixture drop-wise into a second vessel containing the ligation cocktail to achieve dilution without generating large volumes of ligated reaction mixture (FIG. 7). Sufficient time is allowed between each aliquot addition to optimize the intramolecular ligation process for each new aliquot dispensed. Once ligation of the aliquot is complete, the circular DNA is no longer substrate for the enzyme and becomes part of the dilution mix. A second aliquot is then dispensed, and the cycle repeats until all the amplified DNA is dispensed and ligated. This process allows intramolecular ligation to occur without large dilutions of the initial amplification reaction and can incorporate multiple dispensing chambers to allow for simultaneous aliquots to be ligated and to minimize processing time.

Final purification of the product can be streamlined by using permeable membrane-based methods during the reaction process. These membranes permit low molecular weight molecules (salts, unincorporated primers, dNTPs, NTPs and other small molecules) in the amplified DNA reaction mixture to diffuse away while retaining the product. A modification of the hemodialysis process can be used to allow the selective retention of the amplified DNA over other reaction components. Once the reaction is complete, the amplification reaction is pumped from the vessel to a filter comprising membranes with specific molecular weight cut-offs. The DNA is at least partially purified when the smaller reagents diffuse from the reaction across the membrane of these small capillaries. Purified DNA is then either pooled, evaluated for quality and/or dispensed for end-use applications, or directly aliquoted and stored for analysis at a later time. Other embodiments utilize an ultrafiltration purification step which comprises a low-pressure membrane separation process to partition high molecular weight compounds from a feed stream to achieve the desired purification of the final RCA products.

The final product may be analyzed by traditional methods for size, form, contamination, and expression capacity. Gel electrophoresis, sequencing, and biochemical or HPLC analysis is routine. Expression of the final product is tested by transfection into appropriate cells, using standard techniques such as calcium phosphate treatments, electroporation or related techniques.

Administration of the amplified product as a therapeutic compound may include but is not limited to topical applications, intravenous, intramuscular and intra-tissue injections, nasal applications, suppository applications, injections using implanted reservoirs and/or pumps such as Omaya reservoirs, eye-drop applications, orally administered pharmaceuticals, and delivery using ultrasound techniques. Delivery vehicles, for example, may include liposome-mediated or polymer-based transport vehicles as well as a wide variety of capsule or protein-targeting vehicles, and appropriate aerosol carriers for respiratory administration.

### EXAMPLES

### Example 1

### Synthesis and Cell Free Amplification of β-Galactosidase (LacZ-DU)

a) Plasmid-Based Template. pSV-β-Galactosidase vector (Promega Corp. Madison, Wis., USA) was partially digested with EcoR I and Pst I. A fragment of about 4.2 kb containing the CMV promoter, Lac Z ORF and SV40 small T antigen termination sequences (LacZ-DU) was isolated, blunt ended with T4 DNA polymerase and cloned into the Sma I site of pGEM™-7Zf(+) (Promega Corp. Madison, Wis., USA) creating the pGEM-LacZ-DU vector. The LacZ-DU was subsequently excised from pGEM-LacZ-DU with Xba I, gel purified, and circularized using T4 DNA ligase (New England Biolabs, Beverly, Mass., USA) as per manufacturer recommendations.
b) PCR-Based Template. LacZ-DU was amplified from the pVAX™200-GW/lacZ vector (Invitrogen Carlsbad, Calif., USA ) using forward (5′-CGGGATCCGACTCTTCGCGATG TAC-3′) and reverse (5′-CGGGATCCCAGCATGCCTGC-3′) primers containing the BamH I endonuclease recognition site. LacZ-DU was amplified in 50 μl reactions with 200 ng of each primer 10 ng pVAX™200-GW/lacZ vector; 0.2 mM dNTPs; 1× Herculase buffer and 2.5 U Herculase™ polymerase (Stratagene, La Jolla, Calif., USA). Amplification was carried out in a RoboCycler Gradient 40 (Stratagene, La Jolla, Calif., USA) under the following conditions: 2 min at 94° C.; 5 cycles (30 sec 92° C.; 30 sec 40° C., 5 min 72° C.); 25 cycles (30 sec 92° C.; 30 sec 55° C., 5 min 72° C.) and 10 min 72° C. The ˜4.2 kb amplification product was digested with BamH I, gel purified and circularized with T4 DNA ligase.
c) Amplification With Random Hexamers. Reactions containing 10 mM Tris pH 8, 10 ng of circular LacZ-DU and 200 pmol random hexamers (Integrated DNA Technologies, Inc. Coralville Iowa, USA) were heated to 95° C. for 3 min and cooled to room temperature. Phi29 DNA polymerase (10 U, New England Biolabs, Beverly, Mass., USA); 0.2 mM dNTPs and 100 μg/ml BSA were added. Amplification was carried at 30° C. in 50 mM Tris-HCl pH7.5; 10 mM MgCl₂; 10 mM (NH₄)₂SO₄, 4 mM DTT for 16 hr. Following amplification, the phi29 DNA polymerase was heat inactivated (5 min; 65° C.) and the amplified LacZ-DU concatamer was ethanol/salt precipitated and digested with the appropriate endonuclease (Xba I or BamH I) as recommended by the enzyme manufacturer.
d) Amplification With Specific Primers. Using the same conditions as described above, two primers of defined sequence and of opposite complementarity were used to selectively amplify a 2788 bp DU. The defined primers were used at a concentration of 200 pmol each, and consisted of the following sequences: forward primer: 5′-CTGCCAACAAGGTACTCG-3′; reverse primer: 5′-AGCTGCTACTGGGTCTAG-3′. Amplification was carried out in the same manner as previously described and examined by gel electrophoresis to assess successful amplification.
e) Amplification With a Single Sequence-Defined Hexamer. Reactions containing 400 pmol of hexamer 5′-GpGpApApApA-3′ which anneals at 8 different sites on LacZ-DU (4 on the reverse DNA strand at positions 464, 1325, 2579 and 3911; 4 on the forward strand at positions 750, 2871, 3239, and 3260) and 10 ng of circular LacZ-DU were heated to 95° C. for 3 min in 40 mM Tris-HCl pH 8; 10 mM MgCl₂ and cooled to room temperature. Phi29 DNA polymerase (10U, New England Biolabs, Beverly, Mass., USA); 1 mM dNTPs; 5% glycerol; 0.7 U yeast inorganic pyrophosphatase (Sigma, St. Louis, Mo., USA) and 100 μg/ml BSA were added. Amplification was carried out at 30° C. in 50 mM Tris-HCl pH 7.5; 10 mM MgCl₂; 10 mM (NH₄)₂SO₄, 4 mM DTT for 16 hr. Following amplification, the phi29 DNA polymerase was heat inactivated (10 min; 65° C.) and the amplified LacZ-DU concatamer was ethanol/salt precipitated and digested with the appropriate endonuclease (Xba I) as recommended by the enzyme manufacturer. After inactivation of the endonuclease (65° C. for 20 min), circularization of linear LacZ-DU was carried out in ligation buffer (50 mM Tris-HCl pH 7.6; 5 mM MgCl₂; 1 mM ATP; 1 mM DTT; 5% PEG-8000) with about 0.1 Unit/μL T4 DNA ligase (Invitrogen Carlsbad, Calif., USA ) per 100 fmol DNA for 16 hr at about 22° C. (or slightly cooler). Circular LacZ-DU was then ethanol/salt precipitated and resuspended in 10 mM Tris-HCl pH 8.
f) Amplification With a Single Exonuclease-Resistant Sequence-Defined Hexamer. Using the same conditions as above, LacZ-DU was amplified using a defined hexamer with two thiophosphate linkages at the 3′ terminal end (5′GpGpApAp^{s}Ap^{s}A-3′).
g) Amplification With a Single Sequence-Defined Pentamer. Using the same conditions as above, LacZ-DU was amplified using a sequence defined pentamer (5′GpGpApApA-3′) which anneals to LacZ-DU at 19 different sites: 8 on the reverse strand at positions 465, 889, 1326; 1695, 2580, 3666 and 3912; 11 on the forward strand at positions 80, 119, 191, 602, 750, 912, 2871, 3239, 3606, 3815.
h) Amplification With a Single Exonuclease-Resistant and Sequence-Defined Pentamer. Using the same conditions as above, LacZ-DU was amplified using a sequence defined exonuclease resistant pentamer with thiophosphate linkages for the two 3′ terminal nucleotides (5′GpGpAp^{s}Ap^{s}A-3′).
i) Amplification Using a Polymerase Cocktail. Using the same conditions as described in section 1-e, LacZ-plasmid was amplified in the presence of phi29 DNA polymerase and T4 DNA polymerase at ratios ranging from 10:3 to 3:10 (Phi29 enzyme unit:T4 enzyme unit). Optimal amplifications conditions were also shown to work for other templates i.e. Luciferase DU.

### Example2

### Synthesis and Cell Free Amplification of Luciferase (Luc-DU)

The pGL3 vector (Promega Corp. Madison, Wis., USA) was digested with Sal I and Xho I. A fragment of about 2.17 kb containing the SV40 promoter, Luciferase ORF and SV40 small T antigen termination sequences (Luc-DU) was isolated, purified and re-circularized using T4 DNA ligase (Invitrogen, Carlsbad, Calif., USA) as per manufacturer recommendations.
a) Cell Free Amplification. Reactions containing hexamers 5′-ApApTpTp^{s}Gp^{s}C-3′ and 5′-ApGpCpAp^{s}Ap^{s}T-3′ at 400 pmol each and 10 ng/25 μl reaction of circular Luc-DU were heated to 95° C. for 3 min in 40 mM Tris-HCl pH 8; 10 mM MgCl₂ and cooled to room temperature. Phi29 DNA polymerase (10U, New England Biolabs, Beverly, Mass., USA); 1 mM dNTPs (25/25/25/25); 5% glycerol; 0.7 U yeast inorganic pyrophosphatase (Sigma, St. Louis, Mo., USA) and 100 μg/ml BSA were added. Amplification was carried out in 25 μl reaction at 30° C. in 50 mM Tris-HCl pH 7.5; 10 mM MgCl₂; 10 mM (NH₄)₂SO₄, 4 mM DTT for 16 hr. Following amplification, the phi29 DNA polymerase was heat inactivated (10 min; 65° C.) and the amplified Luc-DU concatamers were ethanol/salt precipitated and digested with endonuclease (BamH I) as recommended by the enzyme manufacturer. After inactivation of the endonuclease (65° C. for 20 min), circularization of linear Luc-DU was carried out in ligation buffer (50 mM Tris-HCl pH 7.6; 5 mM MgCl₂; 1 mM ATP; 1 mM DTT; 5% PEG-8000) with 0.1 Unit/μl T4 DNA ligase (Invitrogen Carlsbad, Calif., USA) per μg of DNA for 12-16 hr at 14° C. Circular Luc-DU was then ethanol/salt precipitated and resuspended in 10 mM Tris-HCl pH 8.

### Example 3

### Expression of Amplified DNA in Human Cells

Human A549 lung carcinoma cells (ATCC) were cultured in Dulbecco's modified Eagle's medium (DMEM; Invitrogen Carlsbad, Calif., USA) supplemented with 10% heat-inactivated fetal bovine serum (Hyclone, Logan, Utah, USA), 100 U/ml penicillin, 100 μg/ml streptomycin (Invitrogen Carlsbad, Calif., USA) and incubated at 37° C. in 5% CO₂ environment.

a) DNA Transfection. The day prior to transfection, A549 cells were seeded in 6-well plates at a density of 1×10⁵ cells/ml. GenePORTER 2 transfection reagent (Gene Therapy System, San Diego, Calif., USA) was used for cell transfection as directed by manufacturer. Briefly, cell free amplified DU or parental plasmid DNA (Promega Corp. Madison, Wis., USA) were mixed with 2 μg of carrier pssXE DNA (Chen and McMicken, Gene Ther 10: 1776-1780, 2003) in 50 μl of DNA diluent B and incubated at room temperature for 5 min. DNA solution was then mixed with 7 μl of GenePORTER 2 reagent pre-diluted in 50 μl of serum/antibiotics-free DMEM and incubated at room temperature for an additional 5 min. Meanwhile, A549 cells were washed with PBS and topped with 0.9 ml of serum/antibiotic-free DMEM to which the DNA/GenePORTER solution was subsequently added. Following 4 hr incubation in normal growth environment, the cells were washed with PBS and transfection medium was replaced with normal growth medium supplemented with 10 μl/m of Booster 3 (Gene Therapy System, San Diego, Calif., USA). In experiments using LacZ as reporter, transfections with 50-100 ng of LacZ-DU (˜4.2 kb) were compared to transfections with 100 ng of parental pGEM-LacZ-DU plasmid (˜7.2 kb). In other experiments using the luciferase enzyme as a reporter, 249 ng of Luc-DU (˜2.17 kb) were compared to transfections with 570 ng of pGL3 parental vector (5.01 kb; Promega Corp.).
b) Detection of β-Galactosidase Activity in Transfected Cells. 24 hr post-transfection, A549 cells were rinsed with PBS and lysed in 200/250 ml of 0.1 M phosphate buffer pH 7.5; 0.02% Triton X-100 for 1 hr at room temperature. Cell debris was subsequently removed by centrifugation at 10-13,000 rpm for 5 min. Total protein concentration of cell lysates was determined spectrophotometrically at 280 nm or by modified Bradford assay. 50 μg of total protein were mixed with 0.01 M phosphate buffer pH 7.5; 0.1 M MgCl₂, 45 mM β-mercaptoethanol and 0.01 mM (p-nitrophenyl β-D-galactopyronidase) in 1 ml reactions. After incubation for 1-16 hr at 37° C., absorbance at 410 nm was measured.
c) Detection of Luciferase Activity in Transfected Cells. 24 hr post-transfection, cells were processed as described above. Cell lysates were subsequently adjusted to reflect equal total protein concentration and mixed with an equal volume of 2× Bright-Glo™ substrate (Promega Corp.). Light emission was immediately recorded using a Turner Biosystem 20/20ⁿ luminometer.

### Example 4

### Amplification Conditions

### a) Amplification Buffer.

Glycerol Concentration—Two amplification reactions using 10 ng of Luc-DU template each were set up as described in EXAMPLE 2. In one, addition of glycerol was omitted and replaced with water. Following amplification, DNA was ethanol/salt precipitated and subsequently digested with the appropriate restriction enzyme prior to spectrophotometric quantification at 260 and 280 nm wave lengths. In reactions where glycerol concentration was less than 4% w/v (carry over from the phi29 DNA polymerase and inorganic pyrophosphatase stock solutions) a 5.65% increase in amplification efficiency was observed.
Addition of Molecular Sponge: Two amplification reactions using 10 ng of Luc-DU template each were set up as described in EXAMPLE 2. In one, 5% w/v PEG-8000 was added. Following amplification, DNA was ethanol/salt precipitated and subsequently digested with the appropriate restriction enzyme prior to quantification at 260 and 280 nm wave lengths. No positive effect on amplification yields was recorded.
b) Template Concentration. Amplification reactions containing Luc-DU template concentrations ranging from 1,156 nM to 29 nM were prepared as described in Example 2. Following amplification, DNA was ethanol/salt precipitated and subsequently digested with the appropriate restriction enzyme. Nucleic acid concentrations were determined spectrophotometrically at 260 and 280 nm wave length. A 670-fold amplification was observed using 578 nM template under the amplification conditions delineated above.
c) Deoxyribonucleoside Triphosphate (dNTP) Concentration. Amplification reactions containing 578 nM DU were prepared as described in EXAMPLE 2. Reactions containing dATP, dCTP, dGTP, and dTTP (proportionate ratio of 25/25/25/25) concentrations ranging from 1 mM to 9 mM were tested. Following amplification, DNA was digested with the appropriate restriction enzyme and nucleic acid concentrations were determined spectrophotometrically. Amplification was about 3,000-fold in the presence of 6 mM dNTPs under the amplification conditions delineated above.
d) Customization of dNTP Ratio to Template. Amplification reactions containing 578 nM Luc-DU template were prepared essentially as described in EXAMPLE 2. dATP, dCTP, dGTP and dTTP were individually added to the amplification mix to a final concentration of 9 mM. The ratio of each dNTP with respect to the entire pool was tailored such as to reflect the composition of the luciferase template DNA unit i.e. 27.2% A, 22.3% C, 24.2% G and 26.3% T. Following amplification, DNA was ethanol/salt precipitated and subsequently digested with the appropriate restriction enzyme. Nucleic acid concentrations were determined spectrophotometrically at 260 and 280 nm wavelength. About a 2,780-fold amplification was recorded using 578 nM template under the amplification conditions delineated above.
e) Phi29 DNA Polymerase Concentration. Amplification reactions were prepared as described above in which various phi29 DNA polymerase (New England Biolabs) concentrations ranging from 1 to 20 U/578 nM DNA template were tested in the presence of 9 mM dNTPs. Following amplification, DNA was digested with the appropriate restriction enzyme and nucleic acid concentrations were determined spectrophotometrically. 1 U of phi29 polymerase/578 nM was sufficient to produce a 290-fold amplification, while 20 U of phi29 DNA polymerase amplified 10 ng of template DNA 3,985 times.
t) Sequence-Defined Exonuclease Resistant Hexamer Concentration. Amplification reactions were prepared as described above in which various concentrations of sequence-defined exonuclease resistant hexamers of up to 800 pmol were tested. Following amplification, DNA was digested with the appropriate restriction enzyme and nucleic acid concentrations were determined spectrophotometrically. Increasing primer concentrations by 2 from the initial experimental conditions (EXAMPLE 2) translated into a 1.25-fold increase in amplification yields.
g) One Step Amplification Restriction Enzyme Digestion Reaction. Amplification reactions containing 578 nM Luc-DU template were prepared as described in EXAMPLE 2. Following amplification, phi29 DNA polymerase was heat inactivated at 65° C. for 20 min and 6 U of BamHI enzyme was directly added to the reactions. Following 2 hr at 37° C., the enzyme was heat inactivated and the DNA was ethanol/salt precipitated. Efficiency of DNA digestion was visually assessed by agarose gel electrophoresis as described above.
h) Variable Temperature. Using conditions established above (Example 1), amplification of LacZ-plasmid was carried out at temperatures varying from 25 to 34° C. The optimal temperature was determined based on DNA yields and quality. DNA yields were determined spectrophotometrically while DNA quality was assessed by the determination of error rate using a modified Kunkel method (Kunkel T. A; JBC 260:5787-5796,1985) described in Nelson, J. R. et al., BioTechniques 32:S44-S47, 2002) using full-length LacZ gene (3046 bp) as reporter. Amplifications carried out at 32° C. resulted in a >3300-fold amplification with an error rate of 1.22×10⁻⁶ (a 2.5-fold decrease in the reported error rate of Phi29 DNA polymerase).
i) Amplification With Variable Reaction Times. Using the conditions described in Example 1, amplification of LacZ-plasmid was carried out at 32° C. for variable periods of time (reaction time) ranging from 1 to 16 hr. At each time point the DNA polymerases were heat inactivated at 65° C. for 20 min and DNA was digested with appropriate amounts of restriction endonuclease of directly added to the reactions. The optimal reaction time was determined based on DNA yields and quality. The optimal reaction time resulted in a >3800-fold amplification with a polymerization error rate of 1.7×10⁻⁶.
j) Amplification With Lower Enzyme and Template Concentrations. Using the conditions described in Example 1, LacZ-plasmid and Luciferase DU were amplified in reactions containing half the total enzyme concentration (including Phi29 DNA polymerase, T4 DNA polymerase and Inorganic pyrophosphatase) and 289 nM DNA template. The amplification was carried out for 16 hr at 32° C. Following heat inactivation of the polymerases and subsequent endonuclease digestion of the amplification product, the DNA yields and quality were determined as described above. Half the enzymes and template concentrations from the initial experimental conditions (Example 1) translated into a >5000-fold in amplification yields with a polymerization error rate of 7.7×10⁻⁷ (about a 4 fold decrease in the reported error rate of Phi29 DNA polymerase).
k) Elimination/Reduction of Concatemer Formation During RCA. Using the conditions described in Example 1, LacZ-plasmid was amplified in reactions containing 2U of methylation sensitive SexAI endonuclease in addition to the DNA polymerases. The reaction was carried out at 32° C. for 16 hr. Following amplification/digestion, analysis of the synthesized DNA by agarose gel electrophoresis revealed the presence of discreet linear DNA units.

### Example 5

### Intramolecular Ligation Conditions

a) T4 DNA Ligase. Following restriction enzyme digestion of cell free amplified DNA, heat inactivation of said enzyme and ethanol/salt precipitation of the DNA, the intramolecular ligation (self-ligation) of linear DU was performed in 138 μl and 690 μl reactions respectively containing 700 fmol of DNA in 1× ligation buffer (5% PEG-8000; 50 mM Tris-HCl pH 7.5; 10 mM MgCl₂; 1 mM DTT; 1 mM ATP). Various amounts of T4 DNA ligase (Invitrogen Carlsbad, Calif., USA) were then added (0.6-1.5 U) and ligations were carried out at 14° C. for at least 1 hr. Ligation efficiency was subsequently visually determined by agarose gel electrophoresis of said DNA. 290 μU of T4 DNA ligase per fmol DNA in 690 μl reactions was deemed sufficient for driving the synthesis of monomeric circular DU.
b) Other DNA Ligases. Following linearization of DNA with appropriate restriction enzyme and heat inactivation of said enzyme, intramolecular ligation (self-ligation) of linear DU was performed. Ligations were conducted with either E. coli DNA ligase (NEB) or Taq DNA ligase (NEB) following manufacturer recommendations at 14° C. and 45° C. respectively. Ligation efficiency was subsequently visually determined by agarose gel electrophoresis and compared with T4 DNA ligase products.

### Example 6

### Enrichment for Double Stranded Circular DNA

T4 DNA ligation products were ethanol/salt precipitated and resuspended in 20 μl of Plasmid Safe™ DNase buffer (Epicenter) containing 5U of ATP-dependent DNase as per manufacturer recommendations. Following 30 min incubation at 37° C., DNase enzyme was heat inactivated at 65° C. for 20 min. Reaction efficiency was visually determined by agarose gel electrophoresis revealing the presence of only circular dsDNA which can be re-digested to linear form with appropriate restriction enzymes.

### Example 7

### Expression of Amplified DNA in Mice

Various forms of Luc-DUs were prepared including: linear form, phosphorothioate modified linear form, circular form, circular form treated with Plasmid-Safe™ ATP-dependent DNase (Epicenter, Madison, Wis.). 1 μg of various forms of Luc-DUs were complexed with MAA-PEI at an N:P ratio of 15:1 in PBS at a final volume of 200 μL/mouse. Each group comprising 5 BALB/c mice was injected via tail vein without anesthesia with a single form of MAA-PEI-Luc-DU. Mouse lungs were harvested 24 hours following injection and homogenized in luciferase assay buffer. Luciferase gene expression was measured using Bright-Glo™ kits from Promega according to the manufacturer's instructions.

TABLE 1
Expression of Luc-DU in Mice lungs (ng luciferase/lung;
corrected for background).

| | A | B | C | D | E |
|---|---|---|---|---|---|
| Cage 1 | 0.399 | 0.272 | 0.069 | 0.002 | |
| Cage 2 | | 0.281 | 0.036 | 0.169 | 0.058 |
| Cage 3 | 0.299 | | 0.098 | 0.269 | 0.125 |
| Cage 4 | 0.313 | 0.257 | | | 0.147 |
| Average | 0.34 | 0.27 | 0.07 | 0.15 | 0.11 |
| Std Dev | 0.05 | 0.01 | 0.03 | 0.13 | 0.05 |

A, circular Luc-DU; B, circular Luc-DU treated with DNase; C, linear Luc-DU; D, control plasmid; E: phosphorothioate modified. (average background: 0.05 ng/lung)

In other experiments without MAA-PEI complexed to the DNA, the expression cassette for the luciferase enzyme was change for a stronger promoter and the experiments delineated above were repeated. The results are summarized in the table below.

TABLE 2
Expression of Luc-DU in mice lungs (ng luciferase/lung).

| | Plasmid | Circular | Linear |
|---|---|---|---|
| Cage 1 | 369 | 194 | 392 |
| Cage 2 | 457 | 280 | 407 |
| Cage 3 | 475 | 283 | 587 |
| Average | 433.6667 | 252.3333 | 462 |
| Std Dev | 56.72154 | 50.54041 | 108.5127 |

Experiments using “naked” DNA were also conducted. In this experiment, 15 μg of various DNAs expressing the luciferase gene were introduced into mice by intradermal injections without addition of any carrier. The upper and mid tail sections were chosen as sites of injection. 24 hr post-injection, the animals were sacrificed and the upper and mid tail sections were dissected, homogenized and assayed for expression of luciferase as described above. Results from these experiments are summarized below.

TABLE 3
Expression of Luc-DU in mice skin (ng luciferase/injection site).

| Mouse | Plasmid | Circular | Linear |
|---|---|---|---|
| 1 Upper | 121100 | 279100 | 75090 |
| 1 Mid | 110100 | 187700 | 380400 |
| 2 Upper | 378800 | 784000 | 992800 |
| 2 Mid | 898000 | 169400 | 892400 |
| 3 Upper | 1664000 | 190700 | 22310 |
| 3 Mid | 68260 | 1279000 | 610800 |
| 4 Upper | 233900 | 106500 | 381800 |
| 4 Mid | 133600 | 11120 | 307400 |
| | Plasmid | Circular | Linear |
| Mean | 450970 | 375940 | 457875 |
| SD | 560076.9 | 432518.7 | 352342.5 |
| ng/injection | Plasmid | Circular | Linear |

| | | |
|---|---|---|
| 4509.7 | 3759.4 | 4578.75 |
| 5600.769 | 4325.187 | 3523.425 |

### Example 8

### Genetic Immunization in Mice Against gp160 Protein of HIV-1

A eukaryotic cassette expressing a modified form of human immunodeficiency virus (HIV-1) envelope protein gp160 (gp145ΔCF1; Chakrabarti et al., J. Virol. 2002; 76: 5357-68; Kong et al., J. Virol. 2003; 77: 12764-72) was used as template to generate large quantities of linear gp145ΔCF1-DU expression cassette as described in Example 2.

All animal experiments were approved by the Institutional Review Board for Animal Studies (Baylor College of Medicine; BCM). Supercoiled plasmid DNA and cell-free amplified linear DNA (devoid of plasmid backbone sequences) expressing the gp145ΔCF1 protein were diluted in sterile saline solution and injected into the anterior tibialis muscle of BALB/c mice. Each mouse received injections of 50 μg in each leg at days 0, 14 and 28. Blood samples were collected at days 14 (2 weeks), 28 (4 weeks), 42 (6 weeks) and 56 (8 weeks). Groups of 5 mice were used for each DNA types in addition to a control group injected with saline only. The serum from each blood sample was then used in Enzyme-linked immunosorbant assays (ELISA) to assess the IgG antibody titers against gp160. Briefly, 96 well microtiter plates were coated with a solution of 12.5 ng/μL of purified recombinant HIV-1 _{IIIB} gp160 (Advanced Biotechnologies Inc.) in 50 mM carbonate buffer pH 9.5. The wells were subsequently washed with PBS containing 0.05% Tween 20 (PBS-T) and blocked with a solution of 3% BSA in PBS-T. 100 μL of serially diluted mouse antisera (in 3% BSA) was then applied and plates were incubated overnight at 4° C. The plates were washed with PBS-T and filled with 100 μL of a 1:10,000 dilution of horseradish peroxidase-conjugated goat anti-mouse secondary antibodies (Pierce). Following extensive washing, 50 μL of 3,3′,5,5′-Tetramethybenzidine (Sigma) was added and the calorimetric reaction was stopped with 0.5 N H₂SO₄. The optical density reading was taken at 460 nm.

### Example 9

### Purification of Amplification Product(s)

Once processed into the final form (linear, circular or other), the amplification product is purified by gel filtration chromatography using Sephacryl SF-1000 (GEHC). Briefly, DNA is added onto 1.7 m×1.5 cm Econo-column (Bio-Rad) and eluted with 10 mM Tris Ph 8, 150 mM NaCl, 5 mM EDTA at a flow rate of 1 mL/3.6 min. The DNA content of each elution fraction is monitored by agarose gel electrophoresis and the desired fractions are pooled. The fractions are subsequently concentrated using Centriplus 300 cartridges (Millipore Corp.) are recommended by manufacturer.

Alternatively, anion exchange chromatography using Q sepharose column plumbed to an FPLC or HPLC system can be used. DNA in low salt buffer (LSB; 10 mM Tris-Cl pH 8) would be loaded onto columns. Columns are washed with 10 column volume of LSB. DNA is eluted from the resin with a linear gradient of 10-100% elution buffer (EB; LSB+3 M NaCl) in 20 column volumes. The eluate is monitored at 254 nm and only the peak(s) containing the DNA is collected. Desalting by ultrafiltration using Millipore's Pellicon II UF membranes follows. The DNA quality/integrity is analyzed by agarose gel electrophoresis.

### Example 10

### Quality Assessment of Cell-Free Amplified DNA

Each production lot is assigned an identification number and undergoes a series of test to determine DNA concentration, purity and integrity. DNA concentration is determined my photometric absorbance reading at 260 nm. DNA purity is determined using several methods. Photometric A260/280 ratio, real time PCR (Genomic DNA contamination); HPLC (RNA contamination); micro-BCA test (Protein content, Pierce kit) and LAL test (Endotoxin content, Cambrex kit). In addition a bioburden test is carried out to confirm the sterility of the end product. Each set of test needs to comply with the specification set by the therapeutic industry.

TABLE 3
Typical Quality Testing Assessment Profile

| TEST | METHOD | SPECIFICATION |
|---|---|---|
| DNA concentration | Photometric A260 | 1.0-5.0 mg/ml |
| | Densitometry | |
| Purity | Photometric A260/280 ratio | 1.80-1.98 |
| Appearance | Visual | Clear, colorless |
| | | solution |
| Genomic DNA | Real time PCR | <1% w/w |
| RNA | HPLC/1% agarose gel | <1% w/w |
| | electrophoresis | |
| Protein | Colorimetric (BCA Test) | <100 ng/mg |
| Bioburden | Liquid LB medium | OD 600 = 0 |
| | (16-24 hrs, 37° C.) | |
| Endotoxin | Chromogenic LAL test | <5 EU/mg |
| Identity | Restriction Enzyme | Conforms to specified |
| | Analysis | fragment length |

### Example 11

### Genetic Immunization in Rabbits Against Hepatitis B Virus (HBV)

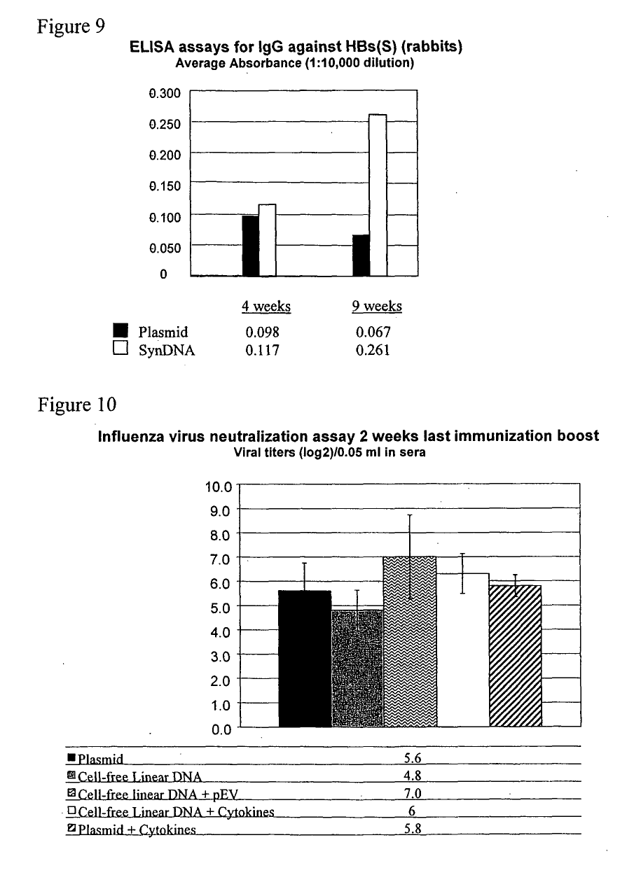

A eukaryotic cassette expressing the Hepatitis B small surface antigen (HBs(S); Davis et al., 1993; Human Mol. Gen. 2: 1847-1851.) was used as template to generate large quantities of linear HBs(S)-DU expression cassette as described in Example 2. Groups of 3 NZ female albino rabbits were immunized via bilateral (hind limb) intramuscular injections on days 0, 28 and 56 with either a total dose of 400 μg of the plasmid each time or the gene equivalent quantity of cell-free amplified linear DNA. Sera from each sample were taken at days 0, 28, 42, 56 and 63 were analyzed with an established ELISA protocol to determine the extent of the humoral immune response. FIG. 9 shows ELISA assay absorbance readings for sera taken from 3 rabbits immunized with either HBs(S) supercoiled plasmid or cell-free HBVs(S)-DU linear DNA for days 28 and 63 (normalized for day 0).

### Example 12

### Genetic Immunization in Mice Against Influenza H1N1 Virus

Five BALB/c mice were utilized in each experiment. All animal experiments were approved by the Institutional Review Board for Animal Studies (Baylor College of Medicine; BCM). Influenza A/Puerto Rico/8/34 (A/PR8; H1N1) was obtained from the Respiratory Pathogens Research Unit, BCM. DNA immunization was conducted as described above using 50 μg of total nucleic acid in PBS. The influenza hemaglutinin open reading frame from viral strain A/PR8/34 (HA) was isolated from pCAG-HA-WPRE plasmid (Garg et al, 2004, J. Immunol. 173(1):550-8) and subcloned into pCMV-MCS (Stratagene) giving pCMV-HA. The CMV-HA expression cassette devoid of plasmid backbone (HA-DU) was amplified as described in Example 2. Animals were given 3 injections at weeks 0, 2 and 6. Five different experiments were conducted. 1) Mice were immunized with 50 μg of pCMV-HA. 2) Mice we immunized with 50 μg of HA-DU. 3) Mice we immunized with a mixture of 25 μg of HA-DU and 25 μg of plasmid DNA devoid of any expression cassette (Empty Vector, pEV). 4) Mice we immunized with a mixture of 16.7 μg of HA-DU and two cytokine-expressing plasmids i.e. 16.7 μg of pCMVi-GMCSF and 16.7 μg of pCAGGSIL12 (Orson et al., 2005, Protection against influenza infection by cytokine enhanced aerosol genetic immunization (In Press). 5) Mice we immunized with a mixture of 16.7 μg of pCMV-HA and two cytokine-expressing plasmids as above.

Eight weeks post immunization, sera samples were taken from each animal and virus neutralization assays were conducted. Sera collected in immunization experiments were heat inactivated (56° C., 30 min) and then assessed in vitro for neutralization efficiency using a standardized microneutralization assay. Briefly, the serum samples were serially diluted 1:2 in duplicate in 96-well, round-bottom tissue culture plates (Falcon 3077) using MEM as the diluent. Then approximately 100 median tissue culture infectious doses (TCID50) of influenza A/PR8 virus was added to each well. A back titration of the test virus was also performed at this time. The plates containing the sera and virus were incubated at 37° C. for 90 minutes, after which the contents of the round-bottom plate were transferred to new plates containing monolayers of Madin Darby canine kidney (MDCK) cells. After overnight incubation at 37° C., the medium from each well was removed and replaced with MEM containing 2 μg/ml of Worthington trypsin (Worthington Biochemical Corp., cat. no. 32C5468), the penicillin and streptomycin, but lacking any serum. Four days later, a 0.5% suspension of chicken red blood cells (rbc) washed and resuspended in PBS was added to each well. When the rbc in the serum control wells formed a tight button, the hemagglutination pattern in each well was read and recorded. Wells with a tight button of rbc were considered to be negative for FV, while those with a diffuse hemagglutination pattern were recorded as positive for virus. FIG. 10 shows virus-neutralization titers recorded as the last dilution in which virus replication was inhibited for the various genetic immunization experiments.

1. A process for producing high-quality nucleic acid in a cell free system, comprising:
(a) combining a circular template in a reaction mixture with one or more primers, which are complementary to at least one strand of the circular template, to form a template-primer complex;
(b) incubating the template-primer complex with at least one high-fidelity nucleic acid polymerase to produce a concatamer comprising tandem units of the circular template; and
(c) cutting the concatamer into smaller fragments comprising at least one delivery unit having a sequence of interest.

2. The process according to claim 1, further comprising:
processing the smaller fragments by the following steps:
filling in or removing the ends of the smaller fragments;
ligating ends of the smaller fragments to produce circularized smaller fragments; and
supercoiling the circularized smaller fragments.

3. The process according to claim 2, the circularized smaller fragments are modified with one or more of the following modifications:
(i) modification at one end with a modified base, a fluorescent tag, a magnetic tag, a radiolabeled tag, or a bio-physical tag;
(ii) modification at an internal base by adding modified bases at step (b);
(iii) modification by adding one or more modified primers at step (a) or (b); and
(iv) conjugation with a nucleic acid, a protein, a peptide, a polymer, or a small molecule.

4. The process according to claim 1, the smaller fragments are modified with one or more of the following modifications:
(i) modification at one end with a modified base a fluorescent tag a magnetic tag, a radiolabeled tag, or a bio-physical tag;
(ii) modification at an internal base by adding modified bases at step (b);
(iii) modification by adding one or more modified primers at step (a) or (b); and
(iv) conjugation with a nucleic acid a protein, a peptide a polymer, or a small molecule.

5. The process according to claim 1, wherein the delivery unit comprises one or more expression cassettes.

6. The process according to claim 1, wherein the high-fidelity nucleic acid polymerase is at least one selected from the group consisting of Phi29 DNA polymerase, Phi29-like DNA polymerase, M2 DNA polymerase, B103 DNA polymerase, GA-1 DNA polymerase, phi-PRD1 polymerase, VENT DNA polymerase, DEEP VENT DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase and a derivative thereof.

7. (canceled)

8. The process according to claim 1, wherein the cutting of the concatamer is accomplished by using a restriction enzyme.

9. A research, diagnostic, or therapeutic composition comprising the delivery unit prepared by the process according to claim 1.

10. A method for preventing or treating a disease or genetic disorder of a human, animal or plant, comprising: administering an effective amount of the composition of claim 9 to a subject in need thereof.

11. The method according to claim 10, wherein the disease is caused by a virus selected from HIV, influenza virus, parainfluenza virus, adenovirus, corona virus, herpes simplex virus, herpes zoster virus, papilloma virus, and rhino virus.

12. The method according to claim 10, wherein the disease is caused by bacteria, mycobacteria, eubacteria or fungi.

13. (canceled)

14. The method of claim 10, wherein the composition is delivered by injection, an aerosol, an oral composition, eyedrops, suppositories, topical ointments, skin patches and soaks, surgically implanted devices, electroporation, pressure-mediated or hydrodynamic injection, ultrasonic nebulization, particle bombardment, or a long-term release system.

15. An apparatus for producing high-quality nucleic acid, comprising:
a reaction vessel having at least one entry port and one exit port that before addition of reaction reagents, is clean, sterile and free of contaminating nucleic acid sequences;
an input device attached to the entry port and which feeds at least one outside component from an outside holding chamber to the reaction vessel;
at least one outside holding chamber connected to the input device;
a means for pumping an outside component from the holding chamber, through the input device, into the reaction vessel;
an export device attached to the exit port of the reaction vessel;
at least one outside receiving chamber connected to the export device;
a means for regulating temperature of the reaction vessel;
a means for monitoring and controlling the progress of a reaction mixture within the reaction vessel; and
a means for mixing the reaction mixture.

16. The apparatus of claim 15 wherein the reaction vessel is made of either a flexible material wherein the means for mixing is applied to the outside of the vessel or a hardened pre-formed material wherein the means for mixing is applied inside the vessel.

17. The apparatus of claims 15 further comprising a programmable computer to automate or remotely monitor, adjust or control one or more of the following: reaction viscosity, nucleic acid concentration, solution turbidity, conductivity, pH, temperature, protein content, endotoxin, bioburden, chemical contaminants, temperature, timing, addition of reagents, mixing, and dispensing of the reaction mixture.

18. (canceled)

19. (canceled)

20. (canceled)

21. The process of claim 1, wherein the circular template is devoid of one or more of: a plasmid replication sequence, a selection sequence, or a marker sequence

22. The process of claim 1, further comprising:
purifying the smaller fragments to meet one or more of the following criteria: level of genomic DNA is <10 ng (based on a human dose), level of RNA is non-detectable on 0.8% agarose gel (based on a human dose), level of bacterial protein is <10 ng (based on a human dose), level of bacterial endotoxin is <1 Unit/kg body weight or <0.1 EU/μg DNA, or is sterile.

23. A method for inducing immunity in a human or an animal, comprising immunizing the human or animal with a composition comprising the smaller fragments prepared by the process of claim 1.

24. The process of claim 1, further comprising addition of one or more of the following proteins: MutS, a single-stranded binding protein, an additional polymerase.

25. The composition of claim 9, wherein the smaller fragments comprise one or more sequences derived from B-galactosidase, luciferase, HIV gp160 envelope protein, hepatitis B small surface antigen, influenza hemaglutinin, influenza neuraminidase, or a cytokine.


SEQUENCE LISTING (4 sequences)

SEQ ID NO: 1 - DNA, 25 bp, Artificial Sequence
  Description of Artificial Sequence Synthetic primer
cgggatccgactcttcgcgatgtac

SEQ ID NO: 2 - DNA, 20 bp, Artificial Sequence
  Description of Artificial Sequence Synthetic primer
cgggatcccagcatgcctgc

SEQ ID NO: 3 - DNA, 18 bp, Artificial Sequence
  Description of Artificial Sequence Synthetic primer
ctgccaacaaggtactcg

SEQ ID NO: 4 - DNA, 18 bp, Artificial Sequence
  Description of Artificial Sequence Synthetic primer
agctgctactgggtctag